(19) United States
(12) Patent Application Publication
(10) Pub. No.: US 2021/0025014 A1
(43) Pub. Date: Jan. 28, 2021

(54) METHODS FOR GENDER IDENTIFICATION AND CULTIVATION OF CANNABIS SEEDS

(71) Applicant: University of Kentucky Research Foundation, Lexington, KY (US)

(72) Inventors: David Hildebrand, Lexington, KY (US); Jia Wen Tan, Lexington, KY (US); Robert Geneve, Lexington, KY (US)

(21) Appl. No.: 16/937,380

(22) Filed: Jul. 23, 2020

Related U.S. Application Data
(60) Provisional application No. 62/877,535, filed on Jul. 23, 2019.

(51) Int. Cl. C12Q 1/6895

(52) U.S. Cl. CPC C12Q 1/6895; C12Q 2600/13

(57) ABSTRACT

Methods for determining a gender of a Cannabis seed include the identification of a Y-chromosome specific marker in a biological sample from a dry Cannabis seed such that the dry Cannabis seed can be identified as being a male or female Cannabis seed based on the presence or absence of the Y-chromosome specific marker. Methods for cultivating a female Cannabis plant include obtaining a biological sample from a dry Cannabis seed; determining the presence or absence of a Y-chromosome specific marker in the dry Cannabis seed sample; identifying the dry Cannabis seed as being a female Cannabis seed based on the absence of the Y-chromosome specific marker; and then germinating the identified female Cannabis seed. Kits for determining the gender of a Cannabis seed are also provided and include a primer pair of amplifying a Y-chromosome specific marker. The Y-chromosome specific marker can be a SCAR119 marker.

### RELATED APPLICATIONS

This application claims priority from U.S. Provisional Application Ser. No. 62/877,535, filed Jul. 23, 2019, the entire disclosure of which is incorporated herein by this reference.

### TECHNICAL FIELD

The presently-disclosed subject matter generally relates to methods for gender identification and cultivation of Cannabis seeds. In particular, certain embodiments of the presently-disclosed subject matter relate to methods for gender identification and cultivation of Cannabis seeds based on the presence or absence of Y-chromosome specific genes such as a SCAR119 marker in a biological sample from a dry Cannabis seed.

### BACKGROUND

Cannabis sativa L. is a plant cultivated worldwide for fiber, medicine, feed, oils, and as an intoxicant. Traditionally, C. sativa is divided into two main types: fiber (industrial hemp) and drug (marijuana). Cannabis plants with low Δ⁹-tetrahydrocannabinol (THC) and a low THC:Cannabidiol (CBD) ratio are classified as industrial hemp. Plants with higher THC≥0.3% are classified as marijuana. In the Cannabis plant, the synthesized and accumulated cannabidiolic acid (CBDA), can be used to generate the neutral form, CBD, through the process of acid decarboxylation. Recent studies have revealed the potential medicinal value of CBD, as CBD-enriched medical Cannabis showed promising results in treatments for intractable epilepsy with only minor and infrequent side effects. Additionally, the potential medical importance of CBD includes treatment of Post-Traumatic Stress Disorder (PTSD) and substance use disorders. CBD and THC accumulate mainly in the glandular trichromes of the plant, and thus female Cannabis plants are valued more than male plants. To avoid the waste of resource on growing male plants, it is therefore crucial to determine the gender as early as possible.

Researchers have revealed the presence of a high number of male-specific markers, which are thought to associated with the Y chromosome, within the dioecious populations. Those male-specific markers include random amplified polymorphic DNA (RAPD) markers and sequence-characterized amplified region (SCAR) markers derived from the former. Many existing molecular markers and Cannabis plant gender kits require DNA from plant leaf or seedling material. Moreover, and although companies and researchers have indicated that plant gender can be determined using young seedlings, no reports to date have examined the use of dry seed DNA for gender identification. Testing of dry seeds would be very advantageous to the hemp industry, however, as genotyping dry seeds can be done at any time and as planting dry seeds is much more efficient than transplanting plants.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes methods for gender identification and cultivation of a Cannabis seed. In some embodiments, a method for determining a gender of a Cannabis seed is provided that comprises the steps of obtaining a biological sample from a dry Cannabis seed; determining a presence or absence of a Y-chromosome specific marker in the biological sample from the dry Cannabis seed; and identifying the dry Cannabis seed as being a male or female Cannabis seed based on the presence or absence of the Y-chromosome specific marker in the biological sample. In some embodiments, the Y-chromosome specific marker is a SCAR119 marker.

In some embodiments, to determine the presence or absence of the Y-chromosome specific marker in a subject, the method includes the steps of isolating an amount of nucleic acid from the biological sample obtained from the dry Cannabis seed and then contacting the isolated nucleic acid with a probe for the Y-chromosome specific marker. For instance, in certain embodiments, determining the presence of the Y-chromosome specific marker comprises a first step of isolating an amount of nucleic acid from the biological sample obtained from the dry Cannabis seed. In some embodiments, that isolated the nucleic acid from the biological sample is then contacted with a Y-chromosome specific primer pair, and the nucleic acid and Y-chromosome specific primer pair is subjected to conditions sufficient to amplify the Y-chromosome specific marker in the biological sample. In some embodiments, the presence or absence of Y-chromosome specific amplification products is then detected.

In some embodiments of the methods described herein that make use of probes and primers to detect a Y-chromosome specific marker, the isolated nucleic acids from the biological sample are amplified by subjecting the nucleic acid to conditions sufficient for carrying out polymerase chain reaction. In some embodiments, the Y-chromosome specific primer pair comprises a first primer having the sequence of SEQ ID NO: 5 and a second primer having the sequence of SEQ ID NO: 6. In some embodiments, the biological sample comprises embryonic tissue.

Further provided, in some embodiments of the presently-disclosed subject matter are methods for cultivating a female Cannabis plant. In some embodiments, a method for cultivating a female Cannabis plant is provided that comprises the steps of obtaining a biological sample from a dry Cannabis seed; determining a presence or absence of a Y-chromosome specific marker in the biological sample from the dry Cannabis seed; identifying the dry Cannabis seed as being a female Cannabis seed based on the absence of the Y-chromosome specific marker in the biological sample; and germinating the identified female Cannabis seed. For example, in some embodiments, a method for cultivating a female Cannabis plant is provided that comprises the steps of obtaining a biological sample from a dry Cannabis seed; determining a presence or absence of a SCAR119 marker in the biological sample from the dry Cannabis seed; identifying the dry Cannabis seed as being a female Cannabis seed based on the absence of the SCAR119 marker in the biological sample; and germinating the identified female Cannabis seed.

To obtain the biological sample in some exemplary methods of cultivating a Cannabis seed, in some embodiments, obtaining the biological sample includes removing a portion of a pericarp and seed coat of the dry hemp seed without affecting a radicle of the dry Cannabis seed, and removing a portion of the embryo of the dry Cannabis seed. In some exemplary cultivation methods, the biological sample comprises embryonic tissue.

With further respect to the determination of the presence or absence of a Y-chromosome specific marker in a biological sample, in some embodiments, determining the presence or absence of the SCAR119 marker comprises isolating an amount of nucleic acid from the biological sample obtained from the dry Cannabis seed; contacting the nucleic acid from the biological sample with a SCAR119 primer pair; subjecting the nucleic acid and SCAR119 primer pair to conditions sufficient to amplify the SCAR119 marker in the biological sample; and detecting the presence or absence of SCAR119 amplification products. In some embodiments, subjecting the nucleic acid to conditions sufficient to amplify the SCAR119 marker comprises subjecting the nucleic acid to conditions sufficient for carrying out polymerase chain reaction, such as polymerase chain reaction using a first primer having the sequence of SEQ ID NO: 5 and a second primer having the sequence of SEQ ID NO: 6.

Still further provided, in some embodiments of the presently-disclosed subject matter are kits for determining a gender of a Cannabis seed. In some embodiments, a kit for determining the gender of a Cannabis seed is provided that comprises a primer pair for amplifying a SCAR119 marker. In some embodiments, the kit further includes reagents for conducting polymerase chain reaction. In some embodiments, the primer pair included in the kit comprises a first primer having the sequence of SEQ ID NO: 5 and a second primer having the sequence of SEQ ID NO: 6.

Further features and advantages of the present invention will become evident to those of ordinary skill in the art after a study of the description, figures, and non-limiting examples in this document.

### BRIEF DESCRIPTION OF THE DRAWINGS

FIG. 1 is an image showing PCR analysis of SCAR323 and MADC2 primer sets, including SCAR323 and MADC2 amplification products from leaf DNA of female (lanes 1-3 and lanes 7-9, respectively) and male (lanes 4-6 and lanes 10-12, respectively) plants from the cultivar ‘GC1’.

FIG. 2 is an image showing PCR analysis of SCAR323 and MADC2 primer sets, including SCAR323 (top) and MADC2 (bottom) amplification products from leaf DNA of female (lanes 2-4) and male (lanes 5-7) plants from the cultivar ‘GC1’.

FIG. 3 is an image showing PCR analysis of MADC2 primers with seed DNA, including MADC2 amplification products from leaf DNA of female (lane 1), male (lane 2) plants and seed DNA (lanes 3-12) from the cultivar ‘GC1’.

FIG. 4 is an image showing PCR analysis of SCAR119 primer set, including SCAR119 amplification products from leaf DNA of female (lanes 1-3) and male (lanes 4-6) plants from the cultivar ‘GC1’.

FIG. 5 includes images showing PCR analysis of SCAR119 primer set with seed DNA, including SCAR119 amplification products from seed DNA of 16 ‘GC1’ seeds, including SCAR119 amplification products from seeds 1-4 (panel A), seeds 5-6 (panel B), seeds 9-12 (panel C), and seeds 13-16 (panel D).

FIG. 6 includes images showing PCR analysis of SCAR119 primer set with leaf DNA from plants established using remnant seeds, including SCAR119 amplification products from leaf DNA of plants established using remnant seeds, including SCAR119 amplification products from plants 1, 4, 7, 8, and 9 (panel A) and from plants 11, 12, 14, 15 (panel B).

### BRIEF DESCRIPTION OF THE SEQUENCE LISTING

SEQ ID NO: 1 is a forward primer for amplifying a MADC2 nucleic acid sequence.

SEQ ID NO: 2 is a reverse primer for amplifying a MADC2 nucleic acid sequence.

SEQ ID NO: 3 is a forward primer for amplifying a SCAR323 nucleic acid sequence.

SEQ ID NO: 4 is a reverse primer for amplifying a SCAR323 nucleic acid sequence.

SEQ ID NO: 5 is a forward primer for amplifying a SCAR119 nucleic acid sequence.

SEQ ID NO: 6 is a reverse primer for amplifying a SCAR119 nucleic acid sequence.

SEQ ID NO: 7 is a nucleic acid sequence of a MADC6 marker.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter includes, among other things, a method for early plant gender determination using nucleic acids (e.g., DNA) obtained from Cannabis seeds and using Cannabis male sex markers. As described in further detail below, subsequent to gender determination, the remnant seeds containing the embryo can be germinated and further cultivated, or can be maintained for later germination and cultivation of a selected gender of Cannabis plants.

In some embodiments of the presently-disclosed subject matter, a method for determining a gender of a Cannabis seed is provided that comprises the steps of: obtaining a biological sample from a dry Cannabis seed; determining a presence or absence of a Y-chromosome specific marker in the biological sample from the dry Cannabis seed; and identifying the dry Cannabis seed as being a male or female Cannabis seed based on the presence or absence of the Y-chromosome specific marker in the biological sample.

The term “Y-chromosome specific marker” is used herein to refer to a molecular marker (i.e., a genetic marker) that is associated with a Y-chromosome present within the genome of an organism and is thus capable of being used to identify the sex of the organism. Such Y-chromosome markers are generally nucleic acid fragments and include, but are not limited to nucleic acid markers such as random amplified polymorphic DNA (RAPD) markers and sequence-characterized amplified region (SCAR) markers derived from those RAPD markers. For example, in some embodiments, a method for determining a gender of a Cannabis seed comprises: obtaining a biological sample from a dry Cannabis seed; determining a presence or absence of a SCAR119 marker in the biological sample from the dry Cannabis seed; and identifying the dry Cannabis seed as being a male or female Cannabis seed based on the presence or absence of the SCAR119 marker in the biological sample.

The term “SCAR” or “Sequence Characterized Amplified Region” is used herein to refer to a marker that comprises a nucleic acid fragment that is amplified from a single genetically defined locus and that is generally identified by PCR amplification using a pair of specific oligonucleotide primers having a specific sequence of approximately 15-30 bases. Such primers can be designed from or as an extended sequence of a RAPD (random amplified polymorphic DNA) fragment. However, by making use of such longer primers for special regions, it is believed that site-competition can be prevented among primers, thus making any results less sensitive to reaction conditions and more reproducible by increasing the specificity. In this regard, as noted above and in some embodiments, gender-specific primers can be implemented and utilized to reliably and reproducibly determine the gender of plants. Such gender-specific SCARS include, but are not limited to, SCAR119. For additional information regarding gender-specific SCARs and associated primers, see, e.g., Törjék, et al. Euphytica, 127(2), 209-218 (2002), which is incorporated herein by reference in its entirety. In some embodiments, the SCAR marker is derived from the nucleotide sequence of a MADC6 marker having the following sequence (GENBANK accession No. AF36495; SEQ ID NO: 7), where the underlined sequences represent the sites of SCAR primers designed in accordance with the presently-disclosed subject matter.

| | |
|---|---|
| 1 | ctagaggccg tggacgcggc ggaggacgat caaacaacaa caaaccgata tgtcagcttt |
| 61 | gcagcagacc tgggcatata gcttcaaaat gttaccacca gtttgacatc tcatttcaag |
| 121 | ctccaggttc cagtcaatta tcggcctcta g |

The terms “nucleic acid” and “nucleic acid sequence” are used interchangeably herein to refer to deoxyribonucleotides or ribonucleotides and polymers thereof in either single or double stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified and/or functional variants thereof and complementary sequences as well as the sequence explicitly indicated. The terms “nucleic acid” or “nucleic acid sequence” can also be used interchangeably with gene or gene fragment, open reading frame (ORF), cDNA, and mRNA encoded by a gene or gene fragment. Given the use of the SCARs described herein, in certain embodiments, the use of the terms “nucleic acid” and “nucleic acid sequence” are particularly used to refer to deoxyribonucleic acid (“DNA”) sequences and fragments thereof.

In some embodiments of the gender determining methods described herein, determining the presence or absence of the Y-chromosome specific marker (e.g., a SCAR119 marker) comprises first isolating an amount of nucleic acid from the biological sample obtained from the dry Cannabis seed and then determining the presence or absence of the Y-chromosome specific marker (e.g., the SCAR119 marker) using a probe for the Y-chromosome specific marker sequence. Nucleic acids isolated from the biological sample can be isolated using any of a number of techniques known to those skilled in the art including through the use of commercially-available DNA extraction kits (e.g., QuickExtract™ Seed DNA Extraction Solution produced by Epicentre, Madison, Wis.).

Once isolated, the nucleic acid from the biological sample obtained from the dry Cannabis seed, can be analyzed for the Y-chromosome specific marker (e.g., the SCAR119 marker) using one or more probes capable of measuring and/or detecting the presence of Y-chromosome specific marker in the isolated nucleic acids. For example, in some embodiments, the term “probe” can be used to describe a DNA fragment that is complementary to the Y-chromosome specific marker sequence of interest (e.g., the SCAR119 sequence) and that, when labeled in some manner, such as with a radioisotope, biotin, fluorescent moiety, antigen, or other detectable modification, can be used to identify a Y-chromosome specific marker present in the nucleic acid isolated from the dry Cannabis seed. In certain embodiments, the probe is a primer useful for affecting amplification of the Y-chromosome specific marker (e.g. ,the SCAR119 marker) in the obtained biological sample.

As used herein, the term “primer” refers to a nucleic acid molecule that can act as a point of initiation of template-directed nucleic acid molecule synthesis under appropriate conditions (for example, in the presence of four different nucleoside triphosphates and a polymerization agent, such as DNA polymerase, RNA polymerase or reverse transcriptase) in an appropriate buffer and at suitable temperatures. As such it is appreciated that certain nucleic acid molecules can serve as a “probe” and as a “primer.” A primer, however, has a 3′ hydroxyl group for extension and is often utilized in pairs (i.e., a primer pair). As used herein, “primer pair” refers to a set of primers that includes a 5′ (upstream) primer that specifically hybridizes with the 5′ end of a sequence to be amplified (e.g. by PCR) and a 3′ (downstream) primer that specifically hybridizes with the complement of the 3′ end of the sequence to be amplified.

In some embodiments of the presently-disclosed subject matter, determining the presence of the Y-chromosome specific marker (e.g., the SCAR119 marker) comprises: isolating an amount of nucleic acid from the biological sample obtained from the dry Cannabis seed; contacting the nucleic acid from the biological sample with a Y-chromosome specific primer pair; subjecting the nucleic acid and Y-chromosome specific primer pair to conditions sufficient to amplify the Y-chromosome specific marker in the biological sample; and then detecting the presence or absence of Y-chromosome specific amplification products. In some embodiments, subjecting the nucleic acid to conditions sufficient to amplify the Y-chromosome specific marker comprises subjecting the nucleic acid to conditions sufficient for carrying out polymerase chain reaction amplification.

As would be recognized by those skilled in the art, “polymerase chain reaction” or “PCR” refers to a reaction in which a specific target nucleic acid sequence is amplified and is commonly utilized for making multiple copies or replicates of a target nucleic acid flanked by primer binding sites. Such reactions generally comprise repetitions of the following steps: (i) denaturing the target nucleic acid, (ii) annealing of primers to the primer binding sites on the target nucleic acid sequence, and (iii) extension of the primers by a nucleic acid polymerase in the presence of nucleoside triphosphates. As the reaction is cycled through different temperatures optimized for each step in a thermal cycler instrument, multiple copies of the target nucleic acid sequence are generated. In some embodiments that make use of PCR to amplify a SCAR119 nucleic acid sequence, the SCAR119 sequence is amplified through the use of a primer pair that comprises a first primer having the sequence of SEQ ID NO: 5 and a second primer having the sequence of SEQ ID NO: 6.

With further respect to polymerase chain reaction, the term “PCR” also encompasses derivative forms of the reaction described above, including but not limited to, reverse transcriptase (RT)-PCR, real-time PCR, nested PCR, quantitative PCR, multiplexed PCR, and the like. RT-PCR indicates a PCR that is preceded by a reverse transcription reaction that converts a target RNA to a complementary single stranded DNA, which is then amplified. For example, where RNA nucleic acid species may be used for detection of certain nucleotide sequences, a DNA copy (cDNA) of the RNA transcripts of interest can be synthesized prior to the amplification step. The cDNA copy can be synthesized by reverse transcription, which may be carried out as a separate step, or in a homogeneous reverse transcription-polymerase chain reaction, a modification of the polymerase chain reaction for amplifying RNA. “Real-time PCR” refers to a PCR in which the amount of reaction product, i.e., the amplicon or amplification product, is monitored as the reaction proceeds. “Nested PCR” refers to a two-stage PCR wherein the amplicon of a first PCR becomes the sample for a second PCR using a new set of primers, at least one of which binds to an interior location of the first amplicon. “Multiplexed PCR” means a PCR wherein multiple target sequences (or a single target sequence and one or more reference sequences) are simultaneously carried out in the same reaction mixture.

In some embodiments, other suitable methods for polynucleotide amplification or methods for detection of amplification products or nucleic acid fragments that are well known to one of ordinary skill in the art may be employed. Other amplification methods may include for example, ligase chain reaction (“LCR”) and rolling circle amplification (“RCA”). In some embodiments, the detection step can comprise gel electrophoresis, capillary electrophoresis, fluorescence resonant energy transfer (FRET), or hybridization to a labeled probe, such as a probe labeled with biotin, a fluorescent moiety, an antigen, a molecular weight tag, a radioactive label, or other detectable modification. In some embodiments, the detection step can comprise the incorporation of a label (such as but not limited to fluorescent or radioactive labels) during an extension reaction. The detection step can further comprise measuring fluorescence, mass, charge, and/or chemiluminescence.

With respect to the biological sample utilized in the methods described herein, the term “biological sample” is used herein to refer to any Cannabis seed fluid or seed tissue potentially including a Y-chromosome specific (e.g., a SCAR119) nucleic acid sequence. In some embodiments, such a biological sample comprises embryonic tissue. In some embodiments, only a small portion of the embryonic tissue is utilized such that the Cannabis seed, upon identification of its gender, can subsequently be selected, germinated, and utilized for producing a gender-specific (e.g., female) Cannabis crop.

Thus, further provided, in some embodiments of the presently-disclosed subject matter, are methods for cultivating a female Cannabis plant. In some embodiments, such methods comprise the steps of: obtaining a biological sample from a dry Cannabis seed; determining a presence or absence of a Y-chromosome specific marker (e.g., a SCAR119 marker) in the biological sample from the dry Cannabis seed; identifying the dry Cannabis seed as being a female Cannabis seed based on the absence of the Y-chromosome specific marker (e.g., the SCAR119 marker) in the biological sample; and germinating the identified female Cannabis seed. In some embodiments, as indicated above, the biological sample comprises embryonic tissue that, in certain embodiments, is obtained by first removing a portion of a pericarp and seed coat of the dry Cannabis seed without affecting a radicle of the dry Cannabis see, and then removing a portion of the embryo of the dry Cannabis seed.

Still further provided, in some embodiments of the presently-disclosed subject matter are kits for determining the gender of a Cannabis seed. In some embodiments, such kits comprise a primer pair for amplifying a Y-chromosome specific marker, such as a SCAR119 marker. In some embodiments, the kits further comprise one or more reagents for conducting polymerase chain reaction, and/or instructions for using the kit. In some embodiments, the primer pair included in the kit comprises a first primer having the sequence of SEQ ID NO: 5 and a second primer having the sequence of SEQ ID NO: 6.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples.

### EXAMPLES

Materials and Methods

Plant Material. Three pre-determined male ‘GC1’ plants and three pre-determined female ‘GC1’ plants were utilized. To ensure the quality and quantity of the DNA, young leaf tissue was obtained from plants that are in the transition of vegetative and flowering state. Young leaf tissue was also collected from plants established from remnant seeds after seed DNA extraction.

Embryo Extraction from Seed. To obtain a small amount of embryo from ‘GC1’ and other cultivar seeds, achenes (seeds) were first placed on a petri dish with weighing paper. Part of the pericarp and seed coat was then removed with a razor or scalpel blade in a location that was not where the radicle was present and was done without damaging the embryo. Once part of the embryo was exposed, a small part was sliced off with a razor or scalpel blade, and the embryo was subsequently placed in a 0.5 ml tube for storage and later cultivation.

DNA Extraction Protocols. A protocol based on a cetyltrimethylammonium bromide (CTAB) method was utilized for DNA extraction. The leaf tissue was first frozen with liquid nitrogen, and the tissue was then ground with mortar and pestle, before being transferred to a 1.5 ml tube. 700 μl CTAB was then added to the tissue and vortexed with the extraction buffer and B-mercaptoethanol having a ratio of 1000 ml/10 ml. The extraction buffer consisted of 0.1M Tris pH 7.5, 0.75M NaCl, 0.01M EDTA, 1% CTAB and ddH₂O. The mixture was then incubated at 65° C. for 60-90 minutes and was inverted every 15 minutes. In a fume hood, 400 μl Chloroform:Isoamyl alcohol (24:1) was then added and the sample was vortexed again. The samples were then spun for 5 minutes at 11×1,000 rpm and the top phase was transferred to a new 1.5 ml tube. 400 μl Chloroform:Isoamyl alcohol (24:1) was then added again and vortexed before spinning for 5 additional minutes and transferring the top phase to new 1.5 ml tube. 300 μl isopropanol was then added, mixed by inversion and then spun for 5 minutes before pouring off the top phase. 500 μl of 70% ethanol was subsequently added, vortexed, and spun for 5 min before pouring off the top phase and air drying the same. After drying, each sample was then resuspended in 35-50 ml Tris (pH 8.0).

For seed extraction, a QuickExtract™ SeedDNA extraction kit was used and the protocol was used. Briefly, 50 μl of the QuickExtract™ DNA extraction solution was first added into the 0.5 ml tube containing seed embryo and was vortex. Each sample was then incubated at 65° C. for 6 minutes, followed by incubation at 95° C. for 2 minutes.

For each extraction, the absorbance of the extracted DNA was measured at 260 nm with a NanoDrop spectrophotometer. The DNA concentration was calculated using the NanoDrop nucleic acid application module. DNA purity was assessed based on 260/280 nm and 260/230 nm absorbance ratio. DNA integrity was evaluated via 0.8% agarose gel electrophoresis.

Primer design. Three male-specific primers were adapted from the literature to test with seed DNA. An adaption of MADC2 from Mandolino et al (1998) and an adaption of SCAR323 and SCAR119 from Torjek et al (2002) was used. Sequences of the primers utilized are shown below:

MADC2

Forward:
(SEQ ID NO: 1)
5′-GTGACGTAGGTAGAGTTGAA-3′
Reverse:
(SEQ ID NO: 2)
5′-GTGACGTAGGCTATGAGAG-3′

This primer set gave a single band for male plants at 390 bp. Two bands at 560 and 870 bp for female or monecious plants.

SCAR323

Forward:
(SEQ ID NO: 3)
5′-GAGCGGACATCATTGCCT-3′
Reverse:
(SEQ ID NO: 4)
5′-ATCACCCCACCGTTTAGG-3′

This primer set gave a single band at 323 bp for male, and no band for female.

SCAR119

Forward:
(SEQ ID NO: 5)
5′-TCAAACAACAACAAACCG-3′
Reverse:
(SEQ ID NO: 6)
5′-GAGGCCGATAATTGACTG-3′

This primer set gave a single band at 119 bp for male, and very faint or no band for female.

PCR analysis. A Polymerase Chain Reaction (PCR) was used that consisted of 3 μl MgCl₂, 1 μl dNTPs, 0.5 units of Taq DNA polymerase, 1 μl forward primer, 1 μl reverse primer, 1 μl of DNA and dH₂O in a 15 μl total reaction volume. For MADC2, the amplifications were carried out in a Bio-Rad thermocycler with an initial step at 94° C. for 2 minutes, 40 cycles of 94° C. for 30 s, 51.2° C. (primer annealing) for 30 s and 72° C. for 1 minute, follow by extension at 72° C. for 4 minutes. For SCAR323, the amplifications were carried out in a Bio-Rad thermocycler with an initial step at 94° C. for 2 minutes, 35 cycles of 94° C. for 30 s, 51-55° C. (primer annealing) for 30 s and 72° C. for 1 minute, follow by extension at 72° C. for 4 minutes. For SCAR119, the amplifications were carried out in a Bio-Rad thermocycler with an initial step at 94° C. for 2 minutes, 40 cycles of 94° C. for 30 s, 50° C. (primer annealing) for 30 s and 72° C. for 1 minute, follow by extension at 72° C. for 4 minutes.

### Example 1—MADC2 and SCAR323

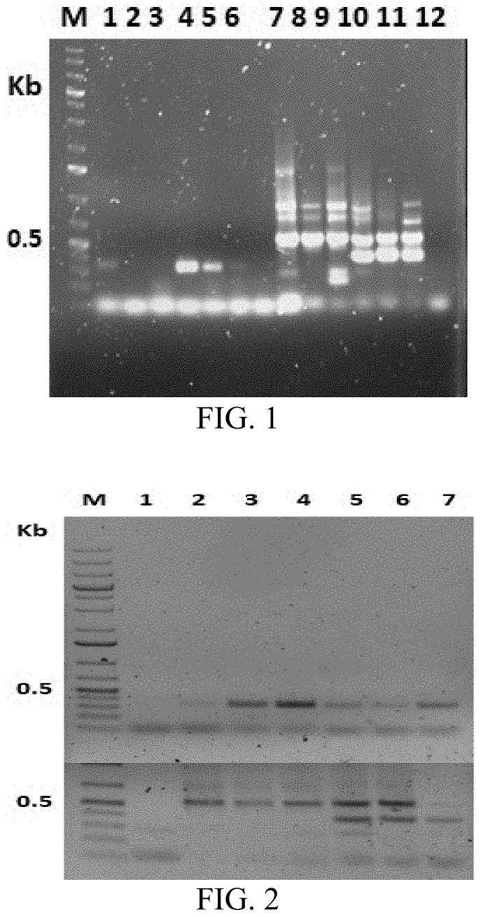

MADC2 primers gave different banding pattern from the literature (FIG. 1), male hemp plant leaf DNA gave a double band at 390 bp and 560 bp, while female leaf DNA gave a single band at 560 bp. This observation is different from the supposed single band at 390 bp for male plants and two bands of 560 and 870 bp for female plants. SCAR323 was more consistent with the literature, as three out of three male hemp plant leaf DNA showed a single band at 323 bp. Only two out of the three female leaf DNA, however, were consistent with literature. While one sample showed a faint band at 323 bp. This first set of DNA was disregarded because of the inconsistency, as potential contamination of PCR could have occurred. Subsequent PCR analysis was carried out to ensure the validity of this analysis.

New sets of leaf DNA were collected to test with SCAR323 and MADC2 primers (FIG. 2). Unlike the last PCR analysis, SCAR323 this time failed to distinguish male plants and female plants. Samples showed a band at 323 bp, indicating that all samples were male. MADC2 showed consistent results with previous sets of DNA. After an initial two rounds of PCR analysis, it was decided to test a new set of DNA with MADC2 due to its consistency.

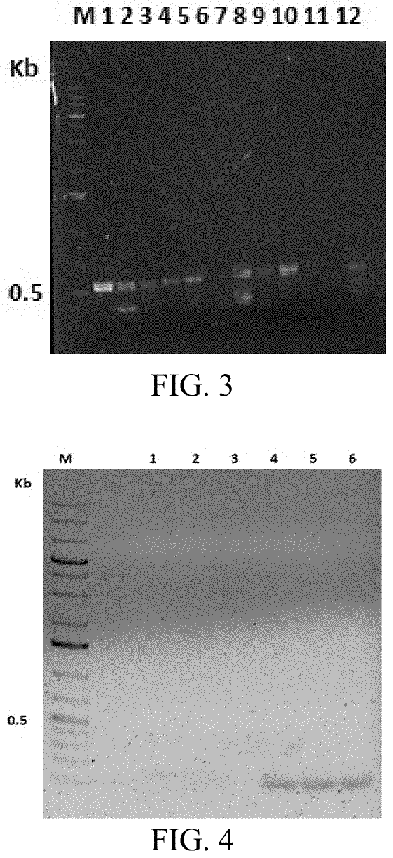

MADC2 was then used to test sets of seed DNA from Cannabis plants from cultivar ‘GC1’ (FIG. 3). The results were still inconsistent with literature but showed consistency with all DNA sets that have been tested before. It was important to note that some seed DNA samples did not give a band, and the problem persisted after several attempts, but with different samples than that shown in FIG. 3. Due to the inconsistency with the literature and the failure to generate clear and convincing data, it was decided to focus on the remaining primer set.

### Example 2—SCAR119 with Leaf DNA

PCR analysis was also performed on primer set SCAR119 (FIG. 4), and three out of three male ‘GC1’ plants leaf DNA samples gave a single band at 119 bp, while three out of three female ‘GC1’ plants leaf DNA samples gave either a faint or no band. Additional PCR analysis were used to confirm this result, the consistency in results and with literature showed potential for seed DNA.

### Example 3—SCAR119 with Seed DNA

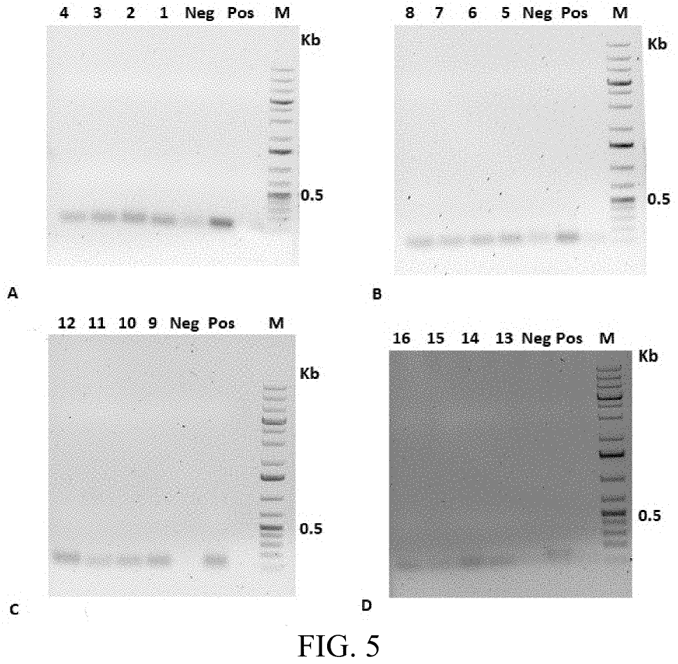

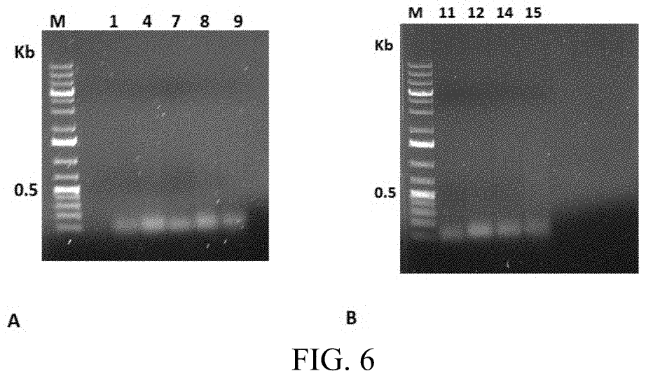

PCR analysis was performed on primer set SCAR119 (FIG. 5), DNA from sixteen seeds have been used. The remnant seeds have been germinated, and it was important to note that although all sixteen (100%) seeds were able to germinate, nine of the sixteen (56%) successfully established into plants. Therefore, nine of the sixteen plants leaf DNA was extracted from three-week-old plants established from remnant seeds were used for PCR analysis (FIG. 6). Additionally, after the extraction, plants were allowed to flower to confirm the PCR results. The result of molecular analysis were found to be concordant with the appearance of male or female flowers (Table 1). Of note, 9 of the 16 seedlings established into full plants, and while DNA analysis showed seeds 1 and 8 to be male plants, the actual plants associated with those seeds appeared to be hermaphrodite, meaning they have both male and female flowers.

TABLE 1
Comparison of molecular analysis and appearance of flowers.

| Seed | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| Seed | ♂ | ♂ | ♂ | ♂ | ♂ | ♂ | ♂ | ♂ | ♂ | ♂ | ♀ | ♂ | ♂ | ♂ | ♂ | ♂ |
| DNA | | | | | | | | | | | | | | | | |
| Leaf | ♂ | — | — | ♂ | — | — | — | ♂ | ♂ | — | ♀ | ♂ | — | ♂ | ♂ | — |
| DNA | | | | | | | | | | | | | | | | |
| Flower | Hermie | — | — | ♂ | — | — | ♂ | Hermie | ♂ | — | ♀ | ♂ | — | ♂ | ♂ | — |

Dash line represent no plants were established from the seed. Hermie:Hermaphrodite, which has both male and female flowers on the same plant.

Discussion of Examples 1-3

In the above-described experiments, out of three primer sets, one proved to work with seed DNA. Although the selected primers sets previously found success in determining the gender of Cannabis sativa plants from leaf DNA, those studies did not consider gender determination using dry seed DNA. As important components such as Cannabidiol (CBD) accumulates mainly in the glandular trichromes of the plant, the determination of plant gender should be done as soon as possible, avoiding the waste of resources.

Between the three primer sets tested, inconsistencies observed with SCAR323 and MADC2 primers could be explained by the complexity of sex determination in hemp, as it is reported to be influenced by autosomic genes. It is also not yet known whether homologs of these two primers can be found in the ‘GC1’ genome. As the complete ‘GC1’ genome sequence remains unknown. As Torjek et al (2002) reported that decamer RAPD primers described as sex-specific markers in different plants are not universal. The inconsistency with the literature but conserved with sample sets likely suggest the targeted region might be located in a different region in Cannabis cultivar ‘GC1’.

The SCAR119 primers showed amplification products in some female controls (FIG. 5), the intensity of the product, however, was consistent with the finding of Torjek et al. (2002), which was consistent with the less intensive female bands in southern hybridizations reported by others. Although the primer has not been cross validated due to the lack of complete genome sequence for ‘GC1’, its consistency in identifying male and female Cannabis plants proved to be more reliable.

The described method of utilizing SCAR119 markers proved to be effective in the discrimination of male Cannabis plants from dry seed DNA, while the remnant seed with embryo still attached was able to germinate. This method was thus an excellent tool for an early screening of plants of important agronomical traits, such as planting all female plants for improved cannabidiol (CBD) production.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

Alghanim, H., & Almirall, J. (2003). Development of microsatellite markers in Cannabis sativa for DNA typing and genetic relatedness analyses. Analytical and Bioanalytical Chemistry, 376(8), 1225-1233.
de Meijer, E. P., Bagatta, M., Carboni, A., Crucitti, P., Moliterni, V. C., Ranalli, P., & Mandolino, G. (2003). The inheritance of chemical phenotype in Cannabis sativa L. Genetics, 163(1), 335-346.
Durand, R., Durand, B., & Jacobs, M. (1990). Sexual determination and sexual differentiation. Critical Reviews in Plant Sciences, 9(4), 295-316.
ElSohly, M. A., & Slade, D. (2005). Chemical constituents of marijuana: the complex mixture of natural cannabinoids. Life sciences, 78(5), 539-548.
Faux, A.-M., Berhin, A., Dauguet, N., & Bertin, P. (2014). Sex chromosomes and quantitative sex expression in monoecious hemp (Cannabis sativa L.). Euphytica, 196(2), 183-197.
Hammond, C. T., & Mahlberg, P. G. (1977). Morphogenesis of capitate glandular hairs of Cannabis sativa (Cannabaceae). American journal of botany, 64(8), 1023-1031.
Hillig, K. W. (2005). Genetic evidence for speciation in Cannabis (Cannabaceae). Genetic Resources and Crop Evolution, 52(2), 161-180.
Kang, H. W., Cho, Y. G., Yoon, U. H., & Eun, M. Y. (1998). A rapid DNA extraction method for RFLP and PCR analysis from a single dry seed. Plant Molecular Biology Reporter, 16(1), 90-90.
Mandolino, G., Carboni, A., Forapani, S., & Ranalli, P. (1998). DNA markers associated with sex phenotype in hemp (Cannabis sativa L.). Proc Bast Fibrous Plants Today and Tomorrow, St Petersburg, September, 28-30.
Mechoulam, R. (1970). Marihuana chemistry. Science, 168(3936), 1159-1166.
O'Connell, B. K., Gloss, D., & Devinsky, O. (2017). Cannabinoids in treatment-resistant epilepsy: a review. Epilepsy & Behavior, 70, 341-348.
Razumova, O. V., Alexandrov, O. S., Divashuk, M. G., Sukhorada, T. I., & Karlov, G. I. (2016). Molecular cytogenetic analysis of monoecious hemp (Cannabis sativa L.) cultivars reveals its karyotype variations and sex chromosomes constitution. Protoplasma, 253(3), 895-901.
Sakamoto, K., Shimomura, K., Komeda, Y., Kamada, H., & Satoh, S. (1995). A male-associated DNA sequence in a dioecious plant, Cannabis sativa L. Plant and Cell Physiology, 36(8), 1549-1554.
Small, E., & Cronquist, A. (1976). A practical and natural taxonomy for Cannabis. Taxon, 405-435.
Techen, N., Chandra, S., Lata, H., ElSohly, M. A., & Khan, I. A. (2010). Genetic identification of female Cannabis sativa plants at early developmental stage. Planta Medica, 76(16), 1938-1939.
Törjék, O., Bucherna, N., Kiss, E., Homoki, H., Finta-Korpelová, Z., Bócsa, I., . . . Heszky, L. E. (2002). Novel male-specific molecular markers (MADC5, MADC6) in hemp. Euphytica, 127(2), 209-218.
Tzadok, M., Uliel-Siboni, S., Linder, I., Kramer, U., Epstein, O., Menascu, S., . . . Granot, D. (2016). CBD-enriched medical Cannabis for intractable pediatric epilepsy: the current Israeli experience. Seizure, 35, 41-44.
Walsh, Z., Gonzalez, R., Crosby, K., Thiessen, M. S., Carroll, C., & Bonn-Miller, M. O., (2017). Medical Cannabis and mental health: A guided systematic review. Clinical psychology review, 51, 15-29.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for determining a gender of a Cannabis seed, comprising:
obtaining a biological sample from a dry Cannabis seed;
determining a presence or absence of a Y-chromosome specific marker in the biological sample from the dry Cannabis seed; and
identifying the dry Cannabis seed as being a male or female Cannabis seed based on the presence or absence of the Y-chromosome specific marker in the biological sample.

2. The method of claim 1, wherein the Y-chromosome specific marker is a SCAR119 marker.

3. The method of claim 1, wherein determining the presence or absence of the Y-chromosome specific marker comprises isolating an amount of nucleic acid from the biological sample obtained from the dry Cannabis seed and contacting the nucleic acid with a probe for the Y-chromosome specific marker.

4. The method of claim 1, wherein determining the presence of the Y-chromosome specific marker comprises:
isolating an amount of nucleic acid from the biological sample obtained from the dry Cannabis seed;
contacting the nucleic acid from the biological sample with a Y-chromosome specific primer pair;
subjecting the nucleic acid and Y-chromosome specific primer pair to conditions sufficient to amplify the Y-chromosome specific marker in the biological sample; and
detecting the presence or absence of Y-chromosome specific amplification products.

5. The method of claim 4, wherein subjecting the nucleic acid to conditions sufficient to amplify the Y-chromosome specific marker comprises subjecting the nucleic acid to conditions sufficient for carrying out polymerase chain reaction.

6. The method of claim 4, wherein the primer pair comprises a first primer having the sequence of SEQ ID NO: 5 and a second primer having the sequence of SEQ ID NO: 6.

7. The method of claim 1, wherein the biological sample comprises embryonic tissue.

8. A method for cultivating a female Cannabis plant, comprising:
obtaining a biological sample from a dry Cannabis seed;
determining a presence or absence of a SCAR119 marker in the biological sample from the dry Cannabis seed;
identifying the dry Cannabis seed as being a female Cannabis seed based on the absence of the SCAR119 marker in the biological sample; and
germinating the identified female Cannabis seed.

9. The method of claim 8, wherein the biological sample comprises embryonic tissue.

10. The method of claim 9, wherein obtaining the biological sample comprises:
removing a portion of a pericarp and seed coat of the dry hemp seed without affecting a radicle of the dry Cannabis seed; and
removing a portion of the embryo of the dry Cannabis seed.

11. The method of claim 8, wherein determining the presence or absence of the SCAR119 marker comprises:
isolating an amount of nucleic acid from the biological sample obtained from the dry Cannabis seed;
contacting the nucleic acid from the biological sample with a SCAR119 primer pair;
subjecting the nucleic acid and SCAR119 primer pair to conditions sufficient to amplify the SCAR119 marker in the biological sample; and
detecting the presence or absence of SCAR119 amplification products.

12. The method of claim 11, wherein subjecting the nucleic acid to conditions sufficient to amplify the SCAR119 marker comprises subjecting the nucleic acid to conditions sufficient for carrying out polymerase chain reaction.

13. The method of claim 11, wherein the primer pair comprises a first primer having the sequence of SEQ ID NO: 5 and a second primer having the sequence of SEQ ID NO: 6.

14. A kit for determining gender of a Cannabis seed, comprising a primer pair for amplifying a SCAR119 marker.

15. The kit of claim 14, further comprising reagents for conducting polymerase chain reaction.

16. The kit of claim 14, wherein the primer pair comprises a first primer having the sequence of SEQ ID NO: 5 and a second primer having the sequence of SEQ ID NO: 6.


SEQUENCE LISTING (7 sequences)

SEQ ID NO: 1 - DNA, 20 bp, Artificial Sequence
  MADC2 forward primer
gtgacgtaggtagagttgaa

SEQ ID NO: 2 - DNA, 19 bp, Artificial Sequence
  MADC2 Reverse Primer
gtgacgtaggctatgagag

SEQ ID NO: 3 - DNA, 18 bp, Artificial Sequence
  SCAR323 Forward Primer
gagcggacatcattgcct

SEQ ID NO: 4 - DNA, 18 bp, Artificial Sequence
  SCAR323 Reverse Primer
atcaccccaccgtttagg

SEQ ID NO: 5 - DNA, 18 bp, Artificial Sequence
  SCAR 119 Forward Primer
tcaaacaacaacaaaccg

SEQ ID NO: 6 - DNA, 18 bp, Artificial Sequence
  SCAR119 Reverse Primer
gaggccgataattgactg

SEQ ID NO: 7 - DNA, 151 bp, Cannabis sativa
ctagaggccgtggacgcggcggaggacgatcaaacaacaacaaaccgatatgtcagctttgcagcagacctgggcatatagcttcaaaatgttaccaccagtttgacatctcatttcaagctccaggttccagtcaattatcggcctctag